(19) United States
(12) Patent Application Publication
(10) Pub. No.: US 2016/0041075 A1
(43) Pub. Date: Feb. 11, 2016

(54) FILTER DEVICE AND MEASURING APPARATUS

(71) Applicant: Murata Manufacturing Co., Ltd., Nagaokakyo-shi (JP)

(72) Inventors: Seiji KAMBA, Nagaokakyo-shi (JP); Takashi KONDO, Nagaokakyo-shi (JP); Hirokazu SETO, Nagaokakyo-shi (JP); Yoshiko MIURA, Nagaokakyo-shi (JP)

(21) Appl. No.: 14/885,410

(22) Filed: Oct. 16, 2015

Related U.S. Application Data
(63) Continuation of application No. PCT/JP2014/064410, filed on May 30, 2014.

(30) Foreign Application Priority Data
May 31, 2013 (JP) 2013-115597

(51) Int. Cl. G01N 1/40; G01J 3/453; C12M 1/26; G01N 21/3586

(52) U.S. Cl. CPC G01N 1/405; G01N 21/3586; G01J 3/453; C12M 33/14

(57) ABSTRACT

A filter device through which a fluid containing a target substance and a coarse substance larger than the target substance is passed to selectively collect the target substance from the fluid. The filter device includes a prefilter that removes the coarse substance and a collection filter that collects the target substance. The prefilter and the collection filter are arranged in series such that the fluid passes through the prefilter and then through the collection filter. The prefilter includes a cavity arrangement structure having a pair of opposing main surfaces and a plurality of cavities extending through both main surfaces.

### CROSS REFERENCE TO RELATED APPLICATIONS

The present application is a continuation of International application No. PCT/JP2014/064410, filed May 30, 2014, which claims priority to Japanese Patent Application No. 2013-115597, filed May 31, 2013, the entire contents of each of which are incorporated herein by reference.

### FIELD OF THE INVENTION

The present invention relates to filter devices and measuring apparatuses.

### BACKGROUND OF THE INVENTION

For example, one method for accurately measuring the amount of a target substance in a fluid (sample), such as PM_{2.5} in air or vesicles in blood, is to selectively collect the target substance to be measured from the fluid before measuring the target substance.

As disclosed in Patent Document 1 (International Publication No. 2014/017430), there is a known method for measuring the presence or amount of a target substance (analyte) in a sample using a cavity arrangement structure as a physical filter. The cavity arrangement structure has a plurality of cavities extending therethrough in a direction perpendicular to the main surfaces thereof. The sample is filtered through the cavity arrangement structure to collect (retain) the target substance on the cavity arrangement structure.

To collect a target substance more selectively and thereby achieve a higher measurement accuracy, it is desirable to reliably remove coarse substances larger than the target substance with a prefilter disposed upstream of a collection filter that collects the target substance to minimize the presence of substances other than the target substance.

In the related art, for example, resin meshes and metal meshes are used as prefilters to remove coarse substances. Other filters such as membrane filters are also used if the coarse substance to be removed is relatively small and cannot be completely removed with the above filters.

Patent Document 1: International Publication No. 2014/017430

### SUMMARY OF THE INVENTION

However, a problem with the use of resin meshes and metal meshes as prefilters is that the accuracy of removal of coarse substances decreases due to variations in processing accuracy and changes in the opening size of resin meshes and metal meshes after deformation under pressure during the passage of a fluid. Another problem is that a filter capable of sufficiently removing coarse substances cannot be fabricated if the coarse substance to be removed is small and the openings of resin meshes and metal meshes cannot be made correspondingly small.

A problem with the use of membrane filters as prefilters is that, although the openings of membrane filters can be made smaller, as described above, the accuracy of removal of coarse substances decreases due to variations in processing accuracy and changes in the opening size of membrane meshes after deformation under pressure during the passage of a fluid. Another problem is that membrane meshes have low filtering efficiency since they have a high pressure loss during the passage of a fluid and may thus be damaged if the fluid is filtered at high flow rate. A further problem is that the use of membrane meshes may result in decreased measurement sensitivity and measurement errors since they have a complicated opening shape and may thus capture a target substance that is smaller than the openings so that it should pass through the openings.

In view of the foregoing background, an object of the present invention is to provide a filter device that can selectively collect an extremely small target substance from a fluid while accurately removing coarse substances larger than the target substance with reduced pressure loss during the passage of the fluid, and to provide a measuring apparatus including such a filter device.

The present invention relates to a filter device through which a fluid containing a target substance and a coarse substance larger than the target substance is passed to selectively collect the target substance from the fluid. The filter device includes a prefilter that removes the coarse substance and a collection filter that collects the target substance.

The prefilter and the collection filter are arranged in series such that the fluid passes through the prefilter and then through the collection filter.

The prefilter includes a cavity arrangement structure having a pair of opposing main surfaces and a plurality of cavities extending through both main surfaces.

The percentage of the open area of the cavities to the area of each main surface of the prefilter including the cavities is preferably 3% to 80%.

The cavities preferably have an aspect ratio of less than 3. The aspect ratio is represented by T/D, where T is the thickness of the cavity arrangement structure, and D is the opening size of the cavities.

The collection filter is preferably a membrane filter. Alternatively, the collection filter preferably includes a stack of cavity arrangement structures shifted from each other, each having a pair of opposing main surfaces and a plurality of cavities extending through both main surfaces.

The present invention also relates to a measuring apparatus including the above-described filter device; and a measuring mechanism that irradiates the collection filter with electromagnetic radiation after the collection filter collects the target substance and that measures the amount of target substance based on the electromagnetic radiation characteristics of the collection filter.

The present invention provides a filter device that can selectively collect an extremely small target substance from a fluid while accurately removing coarse substances larger than the target substance with reduced pressure loss during the passage of the fluid, and also provides a measuring apparatus including such a filter device.

### BRIEF DESCRIPTION OF THE DRAWINGS

FIGS. 1(a) and 1(b) are a series of schematic views illustrating a cavity arrangement structure used in the present invention.

FIGS. 2(a) and 2(b) are a series of schematic views illustrating a filter device according to a first embodiment.

FIGS. 3(a) and 3(b) are a series of schematic views illustrating a filter device according to a second embodiment.

FIG. 4 is a schematic view illustrating a filter device used in the Examples.

FIG. 5 is a series of SEM images of cavity arrangement structures in Example 1.

FIG. 6 is a series of SEM images of a cavity arrangement structure in Comparative Example 1.

FIGS. 7(a) and 7(b) are a series of schematic sectional views illustrating Example 1 and Comparative Example 1.

FIG. 8 is a schematic view showing an example collection filter in the filter device according to the first embodiment.

FIG. 9 is a schematic view showing the configuration of an apparatus used in Example 2.

FIG. 10 is a graph showing the relationship between the percent open area and the relative flow rate of cavity arrangement structures in Example 2.

FIG. 11 is a graph showing the relationship between the thickness and the relative flow rate of cavity arrangement structures in Example 3.

FIG. 12 is a graph showing the frequency characteristics of the change in transmittance in Example 4.

### DETAILED DESCRIPTION OF THE PREFERRED EMBODIMENTS

A filter device according to the present invention is a filter device through which a fluid containing a target substance and a coarse substance larger than the target substance is passed to selectively collect the target substance from the fluid.

The fluid is, for example, a gas or a liquid. The target substance is, for example, an inorganic substance, an organic substance, or a hybrid thereof, a microorganism, or a cell in a fluid. The target substance may be in any form that has a shape in the fluid, such as a solid, liquid, sol, or gel.

Examples of inorganic substances, organic substances, and hybrids thereof in gases include PM_{2.5}, SPM, PM10, and pollen in air. PM_{2.5} (particulate matter) is airborne particulate matter with particle sizes of roughly 2.5 μm or less. To be exact, PM_{2.5} is fine particles that pass through a particle sizer capable of collecting 50% of particles having a particle size of 2.5 μm. PM_{2.5} is thought to be associated with respiratory diseases, cardiovascular diseases, and lung cancer. SPM (suspended particulate matter) is fine particles that pass through a particle sizer capable of collecting 50% of particles having a particle size of 7 μm. PM₁₀ is fine particles that pass through a particle sizer capable of collecting 50% of particles having a particle size of 10 μm.

The filter device according to the present invention includes a prefilter that removes the coarse substance and a collection filter that collects the target substance. The two filters are arranged in series such that the fluid passes through the prefilter and then through the collection filter.

(Prefilter)

The prefilter includes a cavity arrangement structure having a pair of opposing main surfaces and a plurality of cavities extending through both main surfaces. The prefilter may further include other cavity arrangement structures and filters.

(Cavity Arrangement Structure)

The cavity arrangement structure used in the present invention has a pair of opposing main surfaces and a plurality of cavities extending through both main surfaces. For example, the cavities are periodically arranged in at least one direction in the main surfaces of the cavity arrangement structure. All cavities may be periodically arranged. Alternatively, some of the cavities may be periodically arranged, whereas other cavities may be aperiodically arranged, provided that they do not interfere with the advantages of the present invention.

Preferred cavity arrangement structures include quasi-periodic structures and periodic structures. Quasi-periodic structures are ordered structures having no translational symmetry. Examples of quasi-periodic structures include one-dimensional quasi-periodic structures such as Fibonacci structures and two-dimensional quasi-periodic structures such as Penrose structures. Periodic structures are structures having spatial symmetries such as translational symmetry and are classified into one-, two-, and three-dimensional periodic structures according to the number of dimensions of the symmetry. Examples of one-dimensional periodic structures include wire grid structures and one-dimensional diffraction gratings. Examples of two-dimensional periodic structures include mesh filters and two-dimensional diffraction gratings. Preferred periodic structures include two-dimensional periodic structures.

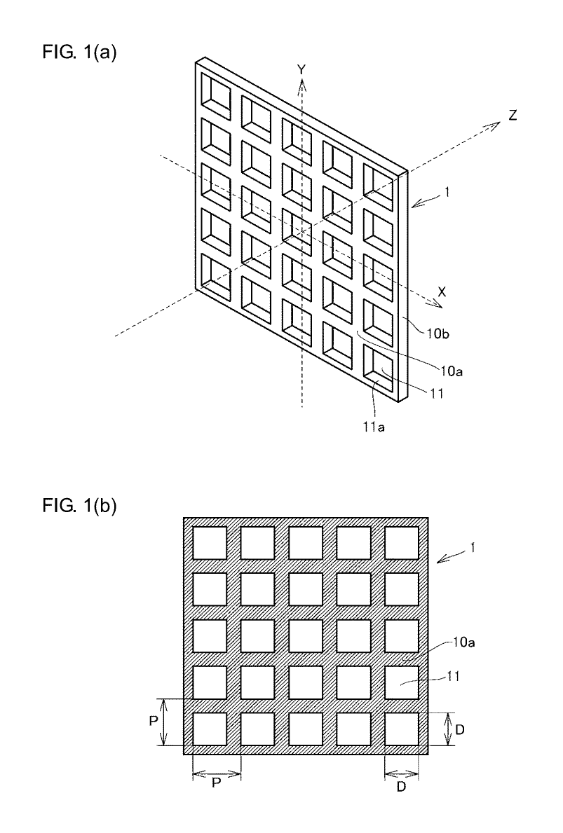

An example two-dimensional periodic structure is a plate-shaped structure (grid structure), as shown in FIG. 1, having cavities arranged in a matrix at predetermined intervals. A cavity arrangement structure 1 shown in FIG. 1(a) is a plate-shaped structure having, as viewed from a main surface 10a thereof, square cavities 11 arranged at regular intervals in two directions parallel to the sides of the squares (i.e., in the vertical and horizontal directions in the figure).

The cavities of the cavity arrangement structure used as the prefilter are preferably sized (e.g., the opening size of the cavities indicated by D in FIG. 1(b)) to allow little or no coarse substance to pass through the cavities and to allow the target substance to pass through the cavities. This allows only the coarse substance to be physically collected (removed) while allowing the target substance to pass through the cavities without being removed.

To increase the flow rate of the fluid through the prefilter, the percentage of the open area of the cavities to the area of each main surface of the prefilter including the cavities (i.e., the percent open area) is preferably 3% or more, more preferably 10% or more. To ensure sufficient strength, the percent open area of the prefilter (cavity arrangement structure) is preferably 80% or less, more preferably 60% or less. The percent open area can be controlled depending on, for example, the opening size of the cavities indicated by D in FIG. 1(b) and the grid pitch of the cavities indicated by P in FIG. 1(b).

The cavities preferably have an aspect ratio of less than 3. The aspect ratio is represented by T/D, where T is the thickness of the cavity arrangement structure, and D is the opening size of the cavities. A T/D of less than 3 effectively reduces the pressure loss during the passage of the fluid through the cavities.

The surface of the cavity arrangement structure is preferably at least partially made of a conductor. The surface of the cavity arrangement structure 1 shown in FIG. 1(a) includes main surfaces 10a, side surfaces 10b, and cavity inner surfaces 11a. The entire cavity arrangement structure may be made of a conductor.

Conductors are materials (substances) that conduct electricity and include metals and semiconductors. Examples of metals include metals capable of binding with the functional groups of compounds having functional groups such as hydroxy, thiol, and carboxy groups; metals capable of being coated with functional groups such as hydroxy and amino groups; and alloys thereof. Specific examples include gold, silver, copper, iron, nickel, chromium, silicon, and germanium, preferably gold, silver, copper, nickel, and chromium, more preferably gold and nickel. Gold and nickel are advantageous if host molecules having thiol groups (—SH groups) are used since the host molecules can be bound to the surfaces of the cavity arrangement structure with the thiol groups. Nickel is advantageous if host molecules having alkoxysilyl groups are used since the host molecules can be bound to the surfaces of the cavity arrangement structure with the alkoxysilyl groups. Examples of semiconductors include Group IV semiconductors (e.g., Si and Ge); compound semiconductors such as Group II-VI semiconductors (e.g., ZnSe, CdS, and ZnO), Group III-V semiconductors (e.g., GaAs, InP, and GaN), Group IV compound semiconductors (e.g., SiC and SiGe), and I-III-VI semiconductors (e.g., CuInSe₂); and organic semiconductors.

(Collection Filter)

“Collecting” the target substance refers to, for example, physically retaining the target substance in the pores of the filter or directly or indirectly depositing the target substance on a surface of the collection filter modified to adsorb the target substance. The prefilter can similarly collect and remove the coarse substance from the fluid.

The pores of the collection filter may have any size suitable for collecting the target substance. For example, the pores of the collection filter are preferably sized to allow little or no target substance to pass physically through the pores. If a cavity arrangement structure is used as the collection filter, the opening size of the cavities of the cavity arrangement structure corresponds to the size of the pores of the collection filter.

Although a cavity arrangement structure having any thickness may be used as the collection filter, a thin cavity arrangement structure is preferably used for reasons of workability if the target substance is extremely small and accordingly the cavities are extremely small. The prefilter does not have to be as thin as the collection filter since the cavities of the cavity arrangement structure used as the prefilter are larger than the cavities of the cavity arrangement structure used as the collection filter. The prefilter, which receives the fluid pressure first, is preferably thicker than the collection filter to achieve a higher mechanical strength.

The collection filter may have a surface modified to adsorb the target substance. If the modified surface can chemically collect the target substance, the pores of the collection filter may be sized to allow the target substance to pass physically through the pores.

For example, the surface modified to adsorb the target substance may be a surface coated with a substance having a high affinity to the target substance. Alternatively, the surface of the collection filter may be modified with host molecules to which the target substance binds. As used herein, the term “host molecules” refers to, for example, molecules to which the target substance can bind specifically. Examples of combinations of host molecules and target substances include antigens with antibodies, sugar chains with proteins, lipids with proteins, low-molecular-weight compounds (ligands) with proteins, proteins with proteins, and single-stranded DNA with single-stranded DNA.

The collection filter may be, for example, a membrane filter or the cavity arrangement structure described above. The collection filter may be composed of a plurality of filters (e.g., membrane filters), a plurality of cavity arrangement structures, or a combination thereof.

(Measuring Apparatus)

A measuring apparatus according to the present invention includes the above-described filter device; and a measuring mechanism that irradiates the collection filter with electromagnetic radiation after the collection filter collects the target substance and that measures the amount of target substance based on the electromagnetic radiation characteristics of the collection filter.

A variety of known mechanisms may be used as the measuring mechanism. Examples of measuring mechanisms include those used for infrared spectroscopy such as Fourier transform infrared spectroscopy (FT-IR) and terahertz time-domain spectroscopy (THz-TDS).

The electromagnetic radiation may be, for example, electromagnetic radiation that can be scattered depending on the structure of the collection filter. Examples of such electromagnetic radiation include radio waves, infrared radiation, visible light, ultraviolet radiation, X-rays, and gamma rays. The frequency of the electromagnetic radiation is preferably, but not limited to, 1 GHz to 1 PHz, more preferably 20 GHz to 200 THz, i.e., terahertz radiation.

The electromagnetic radiation may be, for example, electromagnetic radiation linearly polarized in a predetermined direction (i.e., linearly polarized radiation) or unpolarized electromagnetic radiation (i.e., unpolarized radiation). Examples of linearly polarized electromagnetic radiation include terahertz radiation generated by the optical rectification of light emitted from short-pulsed lasers in electro-optical crystals such as ZnTe crystal, visible light emitted from semiconductor lasers, and electromagnetic radiation emitted from photoconductive antennas. Examples of unpolarized electromagnetic radiation include infrared light emitted from high-pressure mercury lamps and ceramic lamps.

Embodiments of filter devices according to the present invention will now be described with reference to the drawings, where the same reference signs indicate the same or corresponding parts.

### First Embodiment

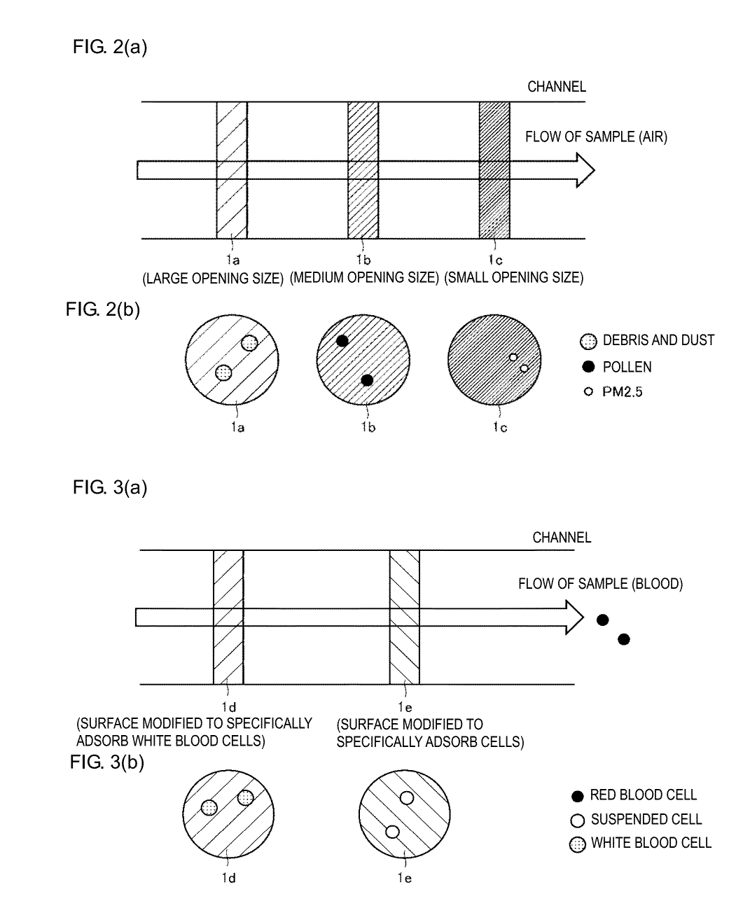

In the filter device according to this embodiment, as shown in FIG. 2(a), a cavity arrangement structure 1a (prefilter) having cavities with a large opening size, a cavity arrangement structure 1b (first collection filter) having cavities with a medium opening size, and a cavity arrangement structure 1c (second collection filter) having cavities with a small opening size are arranged in series inside a channel.

The cavities of the cavity arrangement structure 1a (prefilter) are sized to allow little or no debris and dust (coarse substance) to pass through the cavities and to allow pollen (first target substance) and PM_{2.5} (second target substance) to pass through the cavities. For example, if the cavities of the cavity arrangement structure 1a are regularly arranged in two orthogonal directions, as in the cavity arrangement structure 1 shown in FIG. 1(a), the opening size of the cavities indicated by D in FIG. 1(b) is preferably smaller than or equal to the size of debris and dust (coarse substance) (e.g., the length of the longest straight line joining two points on the surface of the coarse substance), most preferably similar to the size of the coarse substance.

The cavities of the cavity arrangement structure 1b (first collection filter) are sized to allow little or no pollen (first target substance) to pass through the cavities and to allow PM_{2.5} (second target substance) to pass through the cavities. For example, if the cavities of the cavity arrangement structure 1b (first collection filter) are regularly arranged in two orthogonal directions, as in the cavity arrangement structure 1 shown in FIG. 1(a), the opening size of the cavities indicated by D in FIG. 1(b) is preferably smaller than or equal to the size of pollen (first target substance) (e.g., the length of the longest straight line joining two points on the surface of the first target substance), most preferably similar to the size of pollen.

The cavities of the cavity arrangement structure 1c (second collection filter) are sized to allow little or no PM_{2.5} (second target substance) to pass through the cavities. For example, if the cavities of the cavity arrangement structure 1c (second collection filter) are regularly arranged in two orthogonal directions, as in the cavity arrangement structure 1 shown in FIG. 1(a), the opening size of the cavities indicated by D in FIG. 1(b) is preferably smaller than or equal to the size of PM_{2.5} (second target substance) (e.g., the length of the longest straight line joining two points on the surface of the second target substance), most preferably similar to the size of the target substance.

Air (fluid) is then allowed to flow through, in sequence, the cavity arrangement structure 1a, the cavity arrangement structure 1b, and the cavity arrangement structure 1c. As shown in FIG. 2(b), coarse substances such as debris and dust are first captured (removed) by the cavity arrangement structure 1a. Pollen and the like (first target substance) are then captured by the cavity arrangement structure 1b. PM_{2. 5} and the like (second target substance) are then captured by the cavity arrangement structure 1c.

Other examples of collection filters (first and second collection filters) include membrane filters. Membrane filters are suitable if the target substance is extremely small and requires a collection filter having fine pores with a size corresponding to the size of the target substance.

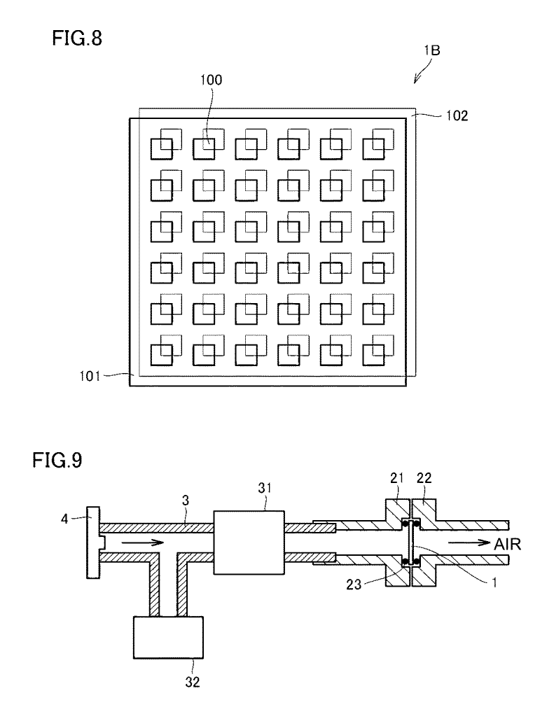

A further example of a collection filter is shown in FIG. 8. This collection filter includes two cavity arrangement structures 101 and 102 stacked such that the cavities overlap only in parts 100. Whereas metal cavity arrangement structures having fine cavities are more difficult to fabricate than membrane filters having fine pores, a collection filter having extremely small cavities can be relatively easily fabricated by stacking a plurality of cavity arrangement structures such that they are shifted from each other to form extremely small cavities defined by the cavities in the cavity arrangement structures. The use of metal cavity arrangement structures as the collection filter also allows the amount of target substance to be sensitively and accurately measured by irradiation with electromagnetic radiation.

In the related art, multi-stage filters as described above have low flow rates because of the high total pressure loss of the individual filters and thus have problems such as hindered fluid flow and decreased processing efficiency. The relative flow rate of a multi-stage filter (i.e., the percentage of the flow rate of a fluid through the multi-stage filter to the flow rate of a fluid without a filter) is approximately equal to the product of the relative flow rates of the filters that constitute the individual stages). For example, the relative flow rate of a two-stage filter composed of a polytetrafluoroethylene (PTFE) membrane filter (average pore size: 3 μm) having a relative flow rate of 11.4% and a PTFE membrane filter (average pore size: 1 μm) having a relative flow rate of 7.2% is as followed: 0.072×0.114=0.0082 (0.82%). This filter hinders the flow of air.

In this embodiment, at least the prefilter is a cavity arrangement structure having a higher relative flow rate than filters such as membrane filters. This reduces the total pressure loss of the multi-stage filter and thus eliminates the problems with the related art, i.e., hindered fluid flow and decreased processing efficiency.

### Second Embodiment

This embodiment differs from the first embodiment in that the collection filter has a surface modified to adsorb the first target substance, and the prefilter has a surface modified to adsorb an impurity or the second target substance and to adsorb little first target substance. The same features as in the first embodiment are not described herein.

Specifically, as shown in FIG. 3(a), a collection filter 1d (prefilter) having a surface modified to specifically adsorb white blood cells (impurity) and a cavity arrangement structure 1e (first cavity arrangement structure) having a surface modified to specifically adsorb suspended cells (first target substance) are first arranged in series inside a channel.

For example, the cavities of the collection filter 1d (prefilter) are sized to allow components having sizes smaller than or equal to the size of suspended cells to pass through the cavities, and the cavities of the cavity arrangement structure 1e (first cavity arrangement structure) are sized to allow components having sizes smaller than or equal to the size of red blood cells to pass through the cavities.

A sample (blood) is then allowed to flow through the cavity arrangement structure 1d and then through the cavity arrangement structure 1e. As shown in FIG. 3(b), white blood cells are first captured by the cavity arrangement structure 1d (second capturing step). Suspended cells are then captured by the cavity arrangement structure 1e (first capturing step). The sample containing red blood cells (blood from which white blood cells and suspended cells have been removed) is drained downstream.

The filter device according to this embodiment is configured such that white blood cells and suspended cells are adsorbed on and collected by surface-modified cavity arrangement structures. This allows the use of a prefilter having an opening size slightly larger than the average size of white blood cells and suspended cells (i.e., a larger opening size than the prefilter in the first embodiment). This further reduces the total pressure loss of the filter device.

The filter devices according to the first and second embodiments can be used to collect various target substances. For example, in addition to the target substances described above, the filter devices according to the first and second embodiments can be used to collect the following target substances.

(i) Research has been devoted to the quantification of exosomes (vesicles) derived from cancer cells in blood as a novel method for cancer diagnosis. There is a need to concentrate exosomes, which have sizes of several hundreds of nanometers, by the filtration of a blood sample through a metal mesh. In view of the components of whole blood, the filter devices according to the foregoing embodiments can be used to extract (collect) only exosomes while removing white blood cells (about 10 μm in size), red blood cells (about 4 μm in size), and other blood cells (about 1 μm in size).

(ii) Norovirus, which cannot be cultivated, cannot be diagnosed until a considerable period of time passes after the illness and the number of virus particles increases. The use of a metal mesh to concentrate a trace amount of virus by filtration allows quick diagnosis without the need for cultivation. For example, there may be a situation where 10 virus particles present in 1 L of sample are concentrated by filtration. The filter devices according to the foregoing embodiments can be used for such selective virus collection.

### EXAMPLES

The present invention is further illustrated by the following examples, although these examples are not intended to limit the scope of the present invention.

### Example 1

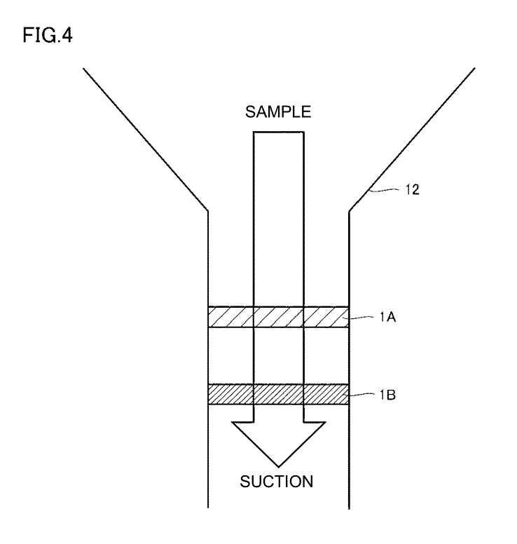

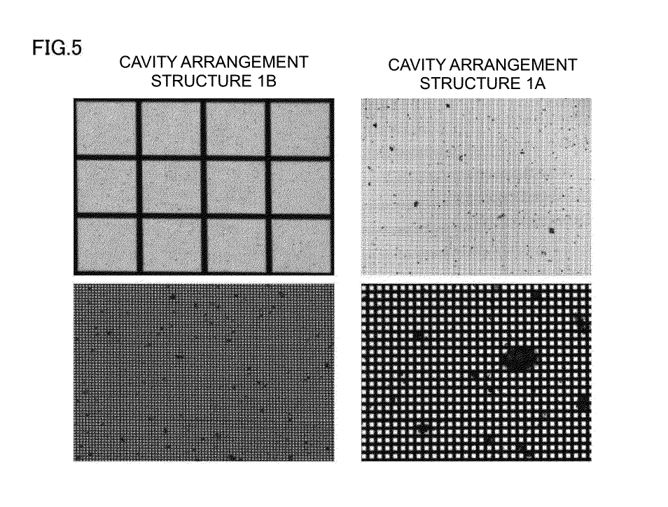

As shown in FIG. 4, a jig 12 having an upward-converging tapered opening was first equipped with a cavity arrangement structure 1A (prefilter) and a cavity arrangement structure 1B (collection filter). The jig 12 equipped with the cavity arrangement structures 1A and 1B was installed outdoors. Air (fluid) was passed through the cavity arrangement structure 1A and then through the cavity arrangement structure 1B (downward in FIG. 4) by suction using a diaphragm pump (suction rate: 11 L/min) for 10 minutes to capture coarse substances other than PM_{2.5} on the cavity arrangement structure 1A and PM_{2.5} (target substance) on the cavity arrangement structure 1B.

The cavity arrangement structures 1A and 1B were nickel plate-shaped structures, as shown in FIG. 1, having square cavities arranged in a square grid pattern in a direction parallel to the main surfaces thereof and having a thickness of 1 to 2 μm. The entire plate-shaped structures were disc-shaped and had an outer diameter of 6 mm. The cavity arrangement structure 1A had a pitch (P in FIG. 1(b)) of 7.1 μm and an opening size (D in FIG. 1(b)) of 4.2 μm. The cavity arrangement structure 1B had a pitch of 2.6 μm and an opening size of 1.8 μm.

FIG. 5 shows scanning electron microscopy (SEM) images of the cavity arrangement structure 1A (right column) and the cavity arrangement structure 1B (left column) after suction filtration in Example 1. The upper images are enlarged images of the lower images. In this example, as shown in FIG. 7(a), air (fluid) was filtered through the two cavity arrangement structures 1A and 1B of different cavity sizes (opening sizes). The results showed that large particles (coarse substances), which are impurities, were captured by the cavity arrangement structure 1A, and only small particles (target substance) were deposited on the cavity arrangement structure 1B, on which no coarse substances were deposited.

### Comparative Example 1

Air was passed through the cavity arrangement structure 1B by suction around 2 p.m. on Apr. 16, 2013 as in Example 1 except that the jig 12 was not equipped with the cavity arrangement structure 1A but only with the cavity arrangement structure 1B.

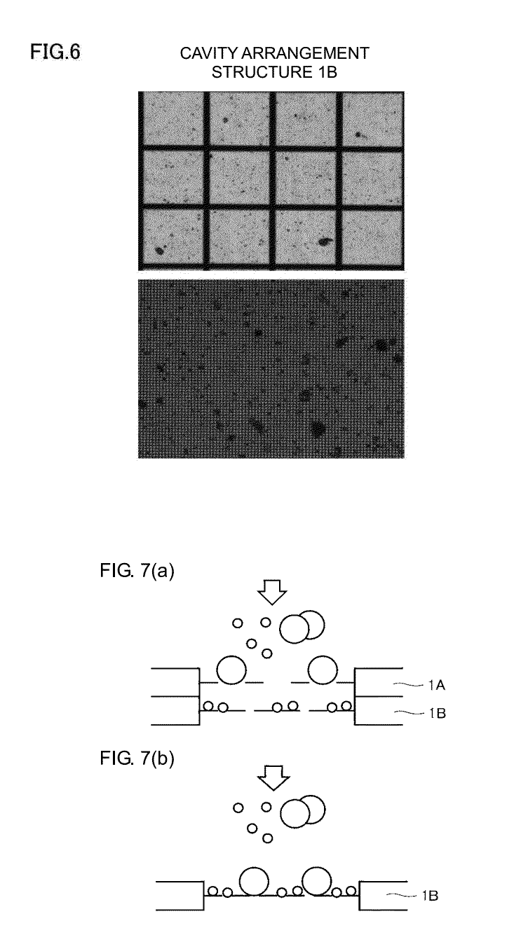

FIG. 6 shows SEM images of the cavity arrangement structure 1B after suction filtration in Comparative Example 1. The upper image is an enlarged image of the lower image. In Comparative Example 1, as shown in FIG. 7(b), the sample (air) was filtered only through the cavity arrangement structure 1B. The results showed that large particles, which are impurities, were also captured by the cavity arrangement structure 1B.

### Example 2

Five nickel plate-shaped structures, as shown in FIG. 1, having square cavities arranged in a square grid pattern in a direction parallel to the main surfaces thereof were first provided as cavity arrangement structures. The entire plate-shaped structures were disc-shaped and had an outer diameter of 6 mm. The cavity arrangement structures had a thickness (T) of 1.0 μm, a cavity pitch (P) of 2.6 μm, and opening sizes (D) of 0.6, 1.0, 1.4, 1.8, and 2.0 μm and thus had different percent open areas (100×D²/P²).

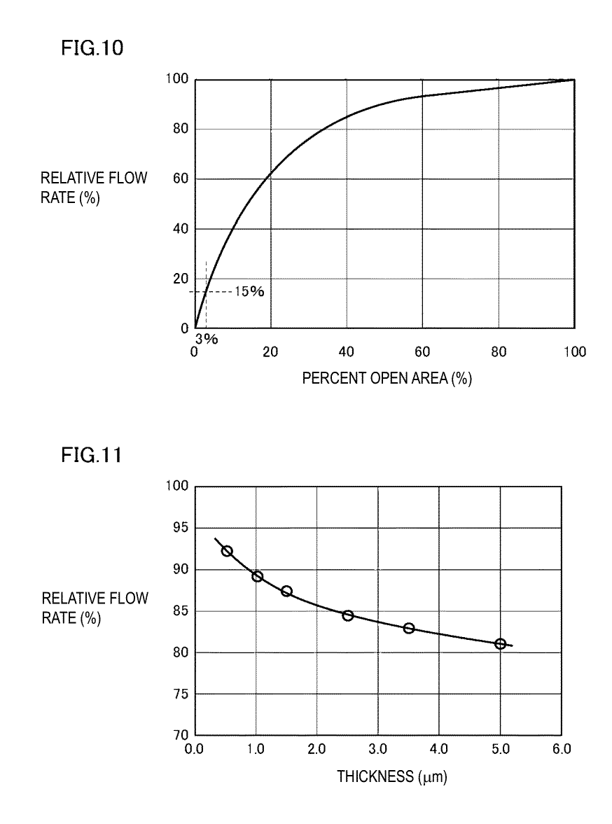

The cavity arrangement structures were attached to the apparatus shown in FIG. 9 using cylindrical jigs 21 and 22 with O-rings therebetween. A small pump 4 (Microblower, Murata Manufacturing Co., Ltd.) mounted on the apparatus shown in FIG. 9 was driven at 15 Vp-p to supply air through a channel pipe 3 to the cavity arrangement structure 1. The flow rate was measured using a flow meter 31. FIG. 10 shows the relationship between the percent open area and the flow rate of the cavity arrangement structure 1 in this example. In FIG. 10, the vertical axis indicates the relative flow rate (%) with respect to the flow rate (100%) of air without the cavity arrangement structure 1 (i.e., at a percent open area of 100%). Although the inner pressure of the channel pipe 3 was also measurable using a pressure gauge 32, it was not measured in this example.

For comparison, similar measurements were performed on commercially available membrane filters made of the materials shown in Table 1, and the relative flow rate (%) was determined. The results are summarized in Table 1.

TABLE 1

| | Average pore size | Porosity | Relative flow rate |
|---|---|---|---|
| Material | (μm) | (%) | (%) |
| Polytetrafluoroethylene | 1.0 | 79 | 7.2 |
| (PTFE) | 3.0 | 83 | 11.4 |
| Polyethylene (PE) | 1.0 | 70 | 3.9 |
| | 5.0 | 72 | 3.1 |

The results in Table 1 show that the flow rate of air through a membrane filter (porosity: 70% to 83%) is about 3% to about 12% of the flow rate of air without the membrane filter. As shown in FIG. 10, the flow rate of air through a cavity arrangement structure having a percent open area of 3% or more is 15% or more of the flow rate of air without the cavity arrangement structure. This demonstrates that a cavity arrangement structure, when used as a filter, can maintain a higher flow rate than a membrane filter.

A cavity arrangement structure having an excessively high percent open area has low mechanical strength since the portion other than the cavities is small. To ensure sufficient strength, the percent open area is preferably 80% or less, more preferably 60% or less.

For example, since a typical blood test uses about 30 mL of blood, it is assumed herein that the load of 30 mL of water (0.03 kg, 2.94×10⁻¹ N) is placed on a nickel cavity arrangement structure having a percent open area of 80% (diameter: 6 mm, pitch (P): 2.6 μm, opening size (D): 2.33 μm, thickness (T): 1 μm). Based on the strength at 1% elongation of nickel (230 to 460 N/mm²), the strength at 1% elongation of the cavity arrangement structure is calculated to be 4.58×10⁻¹ N. This indicates that a nickel cavity arrangement structure having a percent open area of 80% can withstand the load of 30 mL of water (2.94×10⁻¹ N). However, the strength at 1% elongation of a cavity arrangement structure having a percent open area of 90% (diameter: 6 mm, P: 2.6 μm, D: 2.33 μm) is 2.22×10⁻¹ N. This cavity arrangement structure cannot withstand the load of 30 mL of water (2.94×10⁻¹ N) and may thus be deformed. Similarly, the strength of a cavity arrangement structure having a percent open area of 60% or less is calculated to be sufficient to withstand the load of 100 mL of water.

### Example 3

Six cavity arrangement structures were provided that were similar to those of Example 2 except that they had a pitch (P) of 2.6 μm, an opening size (D) of 1.8 μm, and thicknesses (T) of 0.5, 1.0, 1.5, 2.5, 3.5, and 5.0 μm, and the relative flow rate (%) of each cavity arrangement structure was determined as in Example 2. FIG. 11 shows the relationship between the thickness and the relative flow rate of the cavity arrangement structures.

The results in FIG. 11 show that a cavity arrangement structure having an aspect ratio (T/D) within the range tested in this example can maintain a sufficient flow rate compared to those of the membrane filters shown in Table 1. A cavity arrangement structure having a T/D of less than about 3 is technically feasible with the existing cavity arrangement structure fabrication processes. It is desirable to set the thickness (T) within this range since an increase in thickness T within this range results in only a slight decrease in flow rate.

### Example 4

A cavity arrangement structure similar to those of Example 2 except that the opening size (D) was 4 μm was used as a prefilter, and a commercially available PTFE membrane filter (average pore size=1 μm) was used as a collection filter. Air was passed through the prefilter and then through the collection filter using a small pump similar to that of Example 2 (driven at 15 Vp-p) to collect an aerosol with an average particle size of 1 to 4 μm on the membrane filter (collection filter) from the air.

The individual relative flow rates of the prefilter (cavity arrangement structure) and the collection filter (membrane filter) were also determined as in Example 2. The relative flow rate of the prefilter was 78%, whereas the relative flow rate of the collection filter was 7%. In this example, the suction force of the pump was controlled such that the flow rate was 0.4 L/min. Suction was continued at that flow rate for 200 minutes.

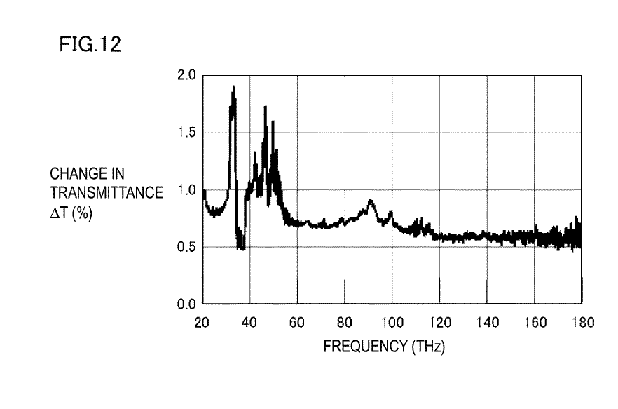

The infrared transmittances of the membrane filter before and after collection were measured by FT-IR, and the change in the infrared transmittance of the membrane filter relative to that before collection (change in transmittance ΔT=transmittance before collection—transmittance after collection) was determined. The frequency characteristics of the change in transmittance are summarized in FIG. 12.

The results shown in FIG. 12 demonstrate that the amount of target substance can be measured from the change in the infrared transmittance of a membrane filter since the transmittance of the membrane filter decreases as more target substance is collected.

The embodiments and examples disclosed herein are to be considered in all respects as illustrative and not restrictive. The scope of the present invention is indicated by the appended claims, rather than by the foregoing description, and all changes that come within the meaning and range of equivalency of the claims are intended to be embraced therein.

### REFERENCE SIGNS LIST

1, 1a, 1b, 1c, 1d, 1e, 1A, 1B, 101, 102 cavity arrangement structure

10a main surface

10b side surface

11 cavity

11a inner surface

12 jig

21, 22 cylindrical jig

23 O-ring

3 channel pipe

31 flow meter

32 pressure gauge

4 small pump

1. A filter device through which a fluid containing a target substance and a coarse substance larger than the target substance can be passed to selectively collect the target substance from the fluid, the filter device comprising:
a prefilter constructed to remove the coarse substance from the fluid; and
a collection filter constructed to collect the target substance from the fluid,
the prefilter and the collection filter being arranged in series such that the fluid passes through the prefilter and then through the collection filter,
the prefilter comprising a cavity arrangement structure having a pair of opposing main surfaces and a plurality of cavities extending through the pair of opposing main surfaces.

2. The filter device according to claim 1, wherein a percentage of an open area of the cavities to an area of each main surface of the prefilter including the cavities is 3% to 80%.

3. The filter device according to claim 2, wherein the cavities have an aspect ratio T/D of less than 3, where T is a thickness of the cavity arrangement structure, and D is an opening size of the cavities.

4. The filter device according to claim 1, wherein a percentage of an open area of the cavities to an area of each main surface of the prefilter including the cavities is 10% to 60%.

5. The filter device according to claim 4, wherein the cavities have an aspect ratio T/D of less than 3, where T is a thickness of the cavity arrangement structure, and D is an opening size of the cavities.

6. The filter device according to claim 1, wherein the cavities have an aspect ratio T/D of less than 3, where T is a thickness of the cavity arrangement structure, and D is an opening size of the cavities.

7. The filter device according to claim 1, wherein the collection filter is a membrane filter.

8. The filter device according to claim 1, wherein the collection filter comprises a stack of arrangement structures shifted relative to each other, each arrangement structure having a pair of opposing main surfaces and a plurality of cavities extending through the pair of opposing main surfaces.

9. The filter device according to claim 1, wherein the cavities are periodically arranged.

10. The filter device according to claim 1, wherein a surface of the cavity arrangement structure is at least partially made of a conductor.

11. The filter device according to claim 1, wherein the entire cavity arrangement structure is made of a conductor.

12. The filter device according to claim 1, wherein the collection filter is modified to adsorb the target substance.

13. The filter device according to claim 1, wherein the prefilter is thicker than the collection filter.

14. The filter device according to claim 13, wherein the collection filter is modified to adsorb the target substance.

15. The filter device according to claim 14, wherein pores of the collection filter are sized to allow the target substance to pass therethrough.

16. A measuring apparatus comprising:
the filter device according to claim 1; and
a measuring mechanism that irradiates the collection filter with electromagnetic radiation after the collection filter collects the target substance and that measures the amount of target substance based on the electromagnetic radiation characteristics of the collection filter.
